(19) United States
(12) Patent Application Publication
(10) Pub. No.: US 2009/0198112 A1
(43) Pub. Date: Aug. 6, 2009

(54) PORTABLE DATA TRANSMITTING DEVICE, AND SYSTEM AND METHOD FOR MANAGING HEAT STRESS USING THE SAME

(75) Inventors: Duck-Gun Park, Daejeon-city (KR); Young Tae Kim, Daejeon-city (KR); Sung Weon Kang, Daejeon-city (KR); Seung Chul Shin, Daejeon-city (KR)

Correspondence Address: AMPACC LAW GROUP, 3500 188th St. SW, Lynnwood, WA, 98037, US

(73) Assignee: Electronics and Telecommunications Research Institute, Daejeon (KR)

(21) Appl. No.: 12/096,371

(22) PCT Filed: Feb. 28, 2006
(86) PCT No.: PCT/KR2006/000695

§ 371 Date: Jun. 5, 2008

(30) Foreign Application Priority Data
Dec. 8, 2005 (KR) 10-2005-0120028
Feb. 24, 2006 (KR) 10-2006-0018437

(51) Int. Cl. A61B 5/00

(52) U.S. Cl. 600/301

(57) ABSTRACT

A highly precise clock synchronization apparatus in a real-time locating system (RTLS), includes an optical transmitting/receiving unit for receiving a clock information frame from a clock synchronization server, converting the received clock information frame in series-parallel, and transmitting/receiving the clock information data and the clock information; an offset estimation unit for detecting a preamble signal and a clock information signal from he series-parallel converted clock information frame, calculating a phase difference value by comparing the detected preamble signal with the detected clock information signal, and outputting an offset value based on the calculated phase difference value; and a clock synchronization unit for updating a local clock value to a time of the clock synchronization server based on the offset value and the clock information frame.

### TECHNICAL FIELD

The present invention relates to a portable data transmission device which monitors extreme heat stress imposed on a user's body, so as to prevent an extremely dangerous situation caused by the extreme heat stress occurring and prepare for an emergency situation, and a system and method of management heat stress.

### BACKGROUND ART

In general, it has been found that heat stress is influenced by temperature, humidity, motion load, and climate acclimatization. In temperatures over 25° C., a human body tries to balance its body temperature by sweating so that the body temperature can be lowered due to vaporization of sweat. This mechanism is influenced by humidity. Thus, in a high humidity conditions, the mechanism becomes less effective. Furthermore, the mechanism is closely influenced by clothing, since air flow is restricted according to the kind of clothing worn.

Both short and long term heat stress is harmful to a human body. Examples of short term impacts include heat stroke, exhaustion, spasmodic motion, and confusion. On the other hand, examples of long term impacts include thermal weakening, high blood pressure, cardiac tissue damage, lack of sexual desire, and impotence.

Representative examples of dangerous occupations in terms of heat stress include police, military personnel, farmers, construction workers, and blast furnace workers.

In addition, firefighters may lose their lives while extinguishing fires such as a forest fire. In some cases, athletes may also lose their lives in the course of training due to heat stress, or due to environmental reasons such as exposure to intense sunlight. Thus, in order to prevent such occupational heat stress and various dangerous heat stress related situations, a method in which the heat stress is measured so that a user can be warned in advance is required.

In particular, heat stress needs to be detected on a regular basis if a workplace is exposed to heat stress, or if the user performs an operation which induces high heat stress.

The conventional wet bulb globe temperature (WBGT) measuring instrument is useful when heat stress has to be directly measured. The WBGT measuring instrument measures four environmentally important key factors, that is, temperature, relative humidity, insolation, and air flow, by comparing a temperature of a freely circulating wet ball with a temperature of a dry ball. A WBGT index is a standard for work in hot environments, ISO7243, and is used as a reference for measuring heat stress.

Conventional heat stress monitoring methods generally use a WBGT system installed in a workplace. Although the WBGT system can measure environmental influence, individual work load and climate acclimatization which are different for each individual cannot be easily taken into account using the WBGT system. In the WBGT system which basically measures environmental influence, the measured heat stress is not actual heat stress imposed on firefighters or blast furnace workers who usually wear protection equipment.

In addition, when a monitoring device is placed in a single position, measuring is not properly performed since thermal exposure is different according to where the monitoring device is located even within the same workplace. In reality, a supervisor has to maintain work stability by relying on a subjective decision by taking the numerous factors mentioned above into account. Thus, only a small number of workers can be supervised by one supervisor. Furthermore, effective preparation and prevention become difficult.

### DETAILED DESCRIPTION OF THE INVENTION

### Technical Problem

The present invention provides a portable data transmission device which wirelessly transmits individual's biometric data and environmental data.

The present invention also provides a heat stress management system which provides an optimum safety guideline in real time by remotely measuring heat stress in real time for each individual.

The present invention also provides a heat stress management method which provides an optimum safety guideline in real time by remotely measuring heat stress in real time for each individual.

### Technical Solution

According to an aspect of the present invention, there is provided a portable data transmission device comprising: a detachable element which is detached from and attached to a user; a biometric data sensor which is connected to the detachable element, and measures user's biometric data; an environmental data sensor which measures data of an environment surrounding the user; and a wireless transmission unit which wirelessly transmits the measured data.

In the aforementioned aspect of the present invention, the portable data transmission device may further comprise: an A/D converter which converts the analog data measured by the sensors to digital signal; and a controller which controls the operation of the wireless transmission unit.

In addition, the sensors and the wireless transmission unit may be disposed on a flexible substrate, and the detachable element may comprise: flexible waterproof cases which encompass the flexible substrate; and an adhesive layer which is coated on one of the outer surfaces of the cases and is detachable from and attachable to a user's skin.

In addition, the cases may be made of a silicon rubber.

In addition, the biometric data may comprise heart rate and motion acceleration, and the environmental data may comprise a temperature and humidity.

According to another aspect of the present invention, there is provided a heat stress management system comprising: a portable data transmission device comprising a detachable element which is detached from and attached to a user, a biometric data sensor which is connected to the detachable element, and measures user's biometric data, an environmental data sensor which measures data of an environment surrounding the user, and a wireless transmission unit which wirelessly transmits the measured data; and a monitoring device which wirelessly receives the biometric data and the environmental data from the wireless transmission unit of the portable data transmission device, and converts the received biometric data, the environmental data, and user's basic data that is input in advance, into a heat stress estimation value, thereby remotely monitoring the user's heat stress.

In the aforementioned aspect of the present invention, the biometric data may comprise heart rate and motion acceleration, the environmental data may comprise temperature and humidity, and the basic data may comprise weight and recent work record.

In addition, the heat stress estimation value may be a work load converted from the heart rate, a WBGT (wet bulb globe temperature) index converted from the temperature and humidity, a climate acclimatization converted from the recent work record, or a physical work degree converted from the weight and motion acceleration.

In addition, the monitoring device may convert the biometric data and the environmental data, which are wirelessly received from the portable data transmission device, and the input basic data into the heat stress estimation value, and may estimate a user's heat stress therefrom, and if the user's heat stress is determined to be extreme, an alarm signal may be automatically generated.

In addition, the monitoring device may be portable.

In addition, the heat stress management system may further comprise a portable data receiving/sending device which wirelessly receives the biometric data and the environmental data from the portable data transmission device, and wirelessly sends the received data to the monitoring device.

In addition, the portable data transmission device, the monitoring device, and the portable data receiving/sending device may communicate with one another wirelessly by using Bluetooth, Zig Bee, a high frequency RF signal, a wireless LAN, a CDMA mobile phone network, a GSM mobile phone network, or a TErrestrial Trunked Radio (TETRA).

According to another aspect of the present invention, there is provided a heat stress management method comprising reviewing user's basic data; wirelessly receiving user's biometric data and environmental data; converting the received data into a heat stress estimation value; estimating a user's heat stress from the heat stress estimation value; and generating an alarm signal if the user's heat stress is extreme.

In the aforementioned aspect of the present invention, the basic data may comprise weight and recent work record, the biometric data may comprise heart rate and motion acceleration, and the environmental data may comprise temperature and humidity.

In addition, the heat stress estimation value may be a work load converted from the heart rate, a WBGT (wet bulb globe temperature) index converted from the temperature and humidity, a climate acclimatization converted from the recent work record, or a physical work degree converted from the weight and motion acceleration.

In addition, the heat stress management method may further comprise automatically writing a report regarding the user's heat stress, if requested.

In addition, the report may comprise the basic data, the biometric data, the environmental data, and the heat stress estimation value.

### ADVANTAGEOUS EFFECTS

According to the present invention, an optimized safety guideline can be provided in real time by remotely measuring heat stress in real time for each individual. Thus, an extremely dangerous situation caused by extreme heat stress imposed on a user's body can be prevented, and an emergency situation can be prepared for. In addition, management efficiency can be maximized since manpower can be effectively managed due to automatic management and reporting.

### DESCRIPTION OF THE DRAWINGS

The above and other features and advantages of the present invention will become more apparent by describing in detail exemplary embodiments thereof with reference to the attached drawings in which:

FIG. 1 is a block diagram of a portable data transmission device according to an embodiment of the present invention;

FIG. 2 is an exploded perspective view illustrating a portable data transmission device according to an embodiment of the present invention;

FIG. 3 is a block diagram of a heat stress management system according to an embodiment of the present invention;

FIG. 4A illustrates a user wearing a heat stress management system according to an embodiment of the present invention;

FIG. 4B is an enlarged view of monitoring device of the heat stress management system of FIG. 4A; and

FIG. 5 is a flowchart illustrating a heat stress management method according to an embodiment of the present invention.

### BEST MODE

Hereinafter, the present invention will be described in detail by explaining exemplary embodiments of the invention with reference to the attached drawings.

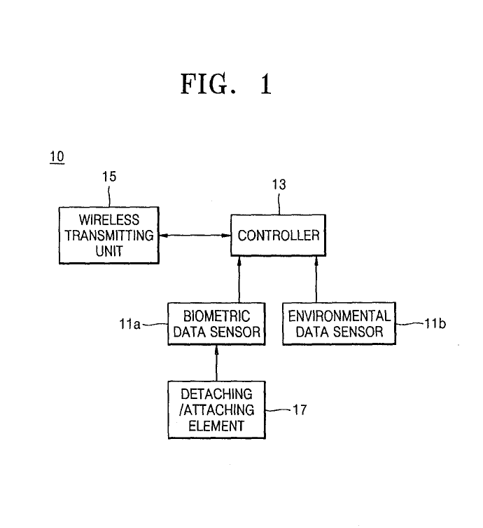

FIG. 1 is a block diagram of a portable data transmission device according to an embodiment of the present invention.

Referring to FIG. 1, a portable data transmission device 10 includes a detachable element 17 which may be detached from and attached to a user, a biometric data sensor 11a which is connected to the detachable element 17 to measure the user's biometric data, an environmental data sensor 11b which measures data of an environment surrounding the user, and a wireless transmission unit 15 which wirelessly transmits the measured biometric data and the environmental data.

Furthermore, the portable data transmission device 10 may include an A/D converter (not shown) which converts the analog data obtained by the sensors 11a and 11b into a digital signal, and a controller 13 which controls the operation of the wireless transmission unit 15. Furthermore, the portable data transmission device 10 may include a memory (not shown) which stores the biometric data and the environmental data, and a power supply unit (not shown) such as a battery.

The biometric data sensor 11a may be formed of one or more sensors provided to measure the biometric data. The biometric data may include heart rate and motion acceleration.

The biometric data sensor 11a may be a well-known sensor for measuring the biometric data, such as heart rate or motion acceleration. For example, an electrocardiogram sensor may be used to measure the heart rate. A two-axis accelerometer (Analog Device, USA) may be used to measure motion acceleration.

The environmental data sensor 11b may be formed of one or more sensors provided to measure environmental data. The environmental data may include temperature and humidity. The environmental data sensor 11b may be a well-known sensor for measuring environmental data, such as temperature or humidity. For example, an on-chip sensor (Sensirion, Swiss), in which a relative humidity sensor is integrated with a temperature sensor, may be used to measure temperature and humidity. When the on-chip sensor is used, a distance between sensors shortens to 0.1 μm.

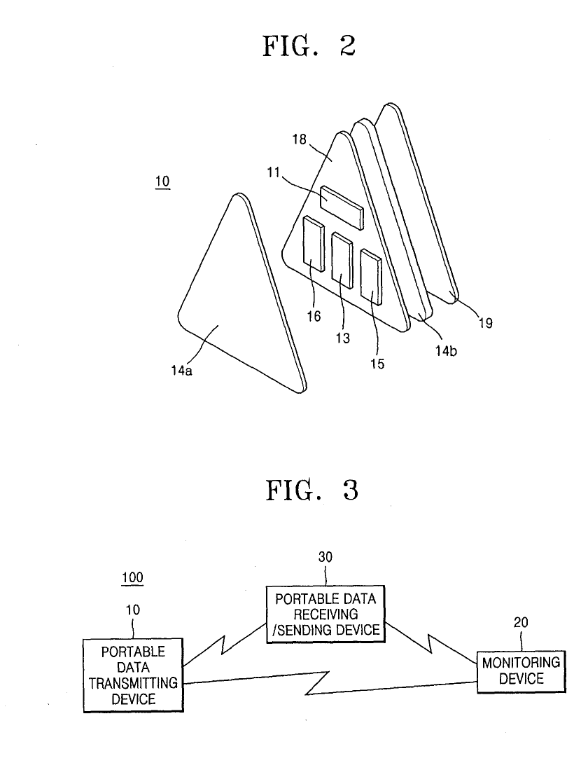

FIG. 2 is an exploded perspective view illustrating a portable data transmission device according to an embodiment of the present invention. In FIG. 2, a sensor 11, a controller 13, a wireless transmission unit 15, and a battery 16 are shown.

Referring to FIG. 2, the sensor 11 and the wireless transmission unit 15 are disposed on a flexible substrate 18. The detachable element 17 of FIG. 1 includes flexible waterproof cases 14a and 14b which encompass the flexible substrate 18, and an adhesive layer 19 which is coated on one of the outer surfaces of the cases 14a and 14b and is detachable from and attachable to a user's skin.

A wearable heat stress sensor that is practically used in a specialized workplace needs to cause minimum restriction, have waterproof properties, be of low weight, be rapid and easy to install, be of low cost, and be applicable to various body shapes.

The portable data transmission device 10 according to an embodiment of the present invention meets all the requirements mentioned above. Specifically, the flexible substrate 18 may be a flexible printed circuit board (FPCB). The flexible waterproof cases 14a and 14b may be made of silicon rubber. The adhesive layer 19 may be a silicon rubber adhesive. By using the FPCB and the silicon rubber packaging, the portable data transmission device 10 can be bent in a flexible manner in order to be attached to a use's chest. In addition, by using the rubber adhesive, the portable data transmission device 10 can be washed by water, and then be reattached to the user's skin.

FIG. 3 is a block diagram of a heat stress management system according to an embodiment of the present invention.

Referring to FIG. 3, a heat stress management system 100 includes a portable data transmission device 10 which is attachable to a user's body, and a monitoring device 20 which wirelessly receives data from the portable data transmission device 10.

The portable data transmission device 10 measures a user's biometric data and environmental data and wirelessly transmits the measured data. Detailed descriptions of the portable data transmission device 10 are the same as described above.

The monitoring device 20 wirelessly receives the biometric data and the environmental data from the wireless transmission unit 15 of the portable data transmission device 10, and converts the received biometric data, the environmental data, and user's basic data that is input in advance, into a heat stress estimation value, thereby remotely monitoring the user's heat stress.

The monitoring device 20 may convert the biometric data and the environmental data, which are wirelessly received from the portable data transmission device 10, and the input basic data into the heat stress estimation value, and may estimate a user's heat stress therefrom. If the user's heat stress is determined to be extreme, an alarm signal may be automatically generated.

The monitoring device 20 may be a computer system existing in a supervisor's domain. Preferably, the monitoring device 20 is portable and carried by the user. For example, the monitoring device 20 may be provided in the form of a wristwatch, a mobile phone, a personal digital assistant (PDA), or a wireless telegraph set. If the user's heat stress is determined to be extreme, the alarm signal may be sent directly to the user.

The biometric data may be the heart rate or motion acceleration of the user. The environmental data may be the temperature or humidity of an environment surrounding the user. The basic data may be a recent weight or recent work record of the user.

The heat stress estimation value may be a work load converted from the heart rate, a wet bulb globe temperature (WBGT) index converted from the temperature and humidity, a climate acclimatization converted from the recent work record, or a physical work degree converted from the weight and motion acceleration.

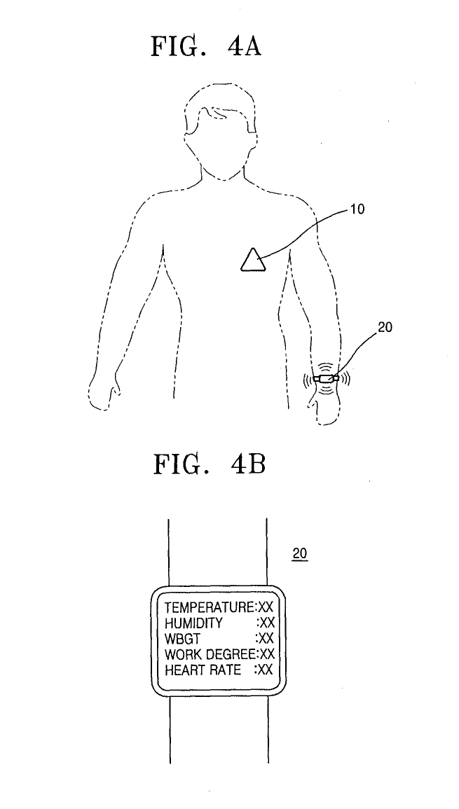

FIG. 4A illustrates a user wearing a heat stress management system according to an embodiment of the present invention. FIG. 4B is an enlarged view of monitoring device of the heat stress management system of FIG. 4A.

Referring to FIG. 4A, a portable data transmission device 10 is placed on a user's chest, measures the user's biometric data and environmental data, and wirelessly transmits the measured data. A monitoring device 20 is placed on a user's wrist in the form of a wristwatch, wirelessly receives the biometric data and the environmental data from the portable data transmission device 10, provides the user with information regarding the biometric data, the environmental data, and the heat stress estimation value, and sends the alarm signal to the user when the user's heat stress is determined to be extreme. Referring to FIG. 4B, the monitoring device 20 has the shape of a wristwatch, and provides information regarding temperature, humidity, WBGT index, work degree, and heart rate.

Referring back to FIG. 3, the heat stress management system 100 may further include a portable data receiving/sending device 30 which relays transmitted data. The portable data receiving/sending device 30 wirelessly receives the biometric data and the environmental data from the portable data transmission device 10, and wirelessly sends the received data to the monitoring device 20. The portable data receiving/sending device 30 may be provided in the form of a wristwatch, a mobile phone, a PDA, or a wireless telegraph set.

The portable data transmission device 10, the monitoring device 20, and the portable data receiving/sending device 30 may wirelessly communicate with one another by using Bluetooth, Zig Bee, a high frequency RF signal, a wireless LAN, a CDMA mobile phone network, a GSM mobile phone network, or a TErrestrial Trunked Radio (TETRA).

The above methods may vary according to an environment of the heat stress management system. For example, the portable data transmission device 10 may include a Bluetooth or Zig Bee module so as to transmit data to a main server via a user's wireless telegraph set or a mobile phone. In addition, the portable data transmission device 10 may include a CDMA module so as to transmit the data directly to the main server.

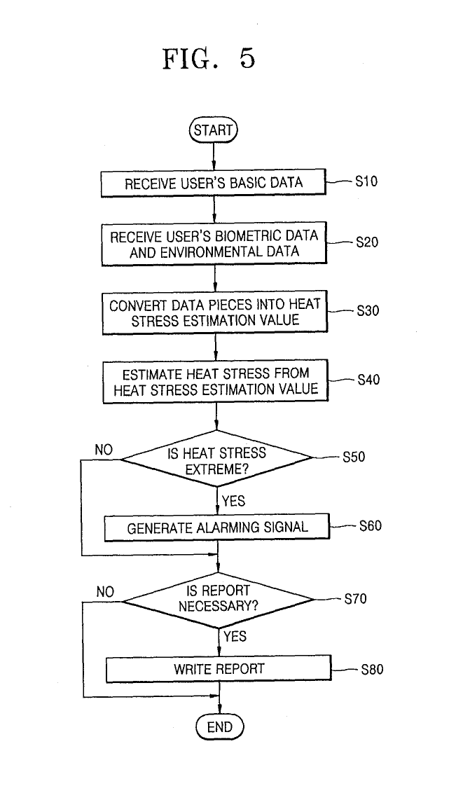

FIG. 5 is a flowchart illustrating a heat stress management method according to an embodiment of the present invention.

Referring to FIG. 5, a heat stress management method includes receiving user's basic data (operation S10), wirelessly receiving user's biometric data and the environmental data (operation S20), converting the data into a heat stress estimation value (operation S30), estimating a user's heat stress from the heat stress estimation value (operation S40), and determining whether the user's heat stress is extreme (operation S50). If the user's heat stress is determined to be extreme, an alarm signal is generated (operation S60).

The basic data may be a recent weight or recent work record of the user. The user's biometric data may be a heart rate or motion acceleration of the user. The environmental data may be a temperature or humidity of an environment surrounding the user.

The heat stress estimation value may be a work load, a WBGT index, a climate acclimatization, or a physical work degree. The conventional heat stress monitoring methods have used only the WBGT index for environmental temperature and humidity, resulting in inaccurate monitoring.

The work load of the heat stress estimation value may be converted from the heart rate. A lead electrocardiograph (ECG) may be used to measure the heart rate. A method of estimating the work load from the heart rate may use a well-known estimation method (Shaver, Essentials of Exercise Physiology, Section Three, The Heart and Exercise, Burgess Publishing Company, pp. 74-93, 1981).

The WBGT index of the heat stress estimation value may be calculated from the temperature or the humidity. An optimum WBGT index may be attained for each individual in real time by obtaining temperature and relative humidity data from a sensor attached to a user's body. For example, an on-chip sensor (Sensirion, Swiss), in which a relative humidity sensor is integrated with a temperature sensor, may be used to measure the temperature and the humidity. A relative humidity of gas is highly dependent on its temperature. Thus, a humidity sensor has to be used at the same temperature as the air in which the relative humidity will be measured.

The WBGT is influenced by air flow (wind), air temperature, humidity, and insolation. The WBGT may be estimated using the following Formula in an environment providing normal insulation and mild wind (American College of Sports Medicine, Med. J. Aust., Dec. 876, 1984).

WBGT=0.567×Ta+0.393×e+3.94 [Formula 1]

Here, Ta is a temperature (° C.) of a wet ball, and e is a steam pressure, or humidity (hPa) of water.

The steam pressure can be calculated from temperature and relative humidity using Formula 2.

e=rh/100×6.105×exp(17.27×Ta/(237.7+Ta)) [Formula 2]

Here, rh is relative humidity (%).

The climate acclimatization of the heat stress estimation value may be calculated from the recent work record. The climate acclimatization is defined as a process in which the user slowly acclimatizes to a climate for one or two weeks. Once the user acclimatizes to the climate, it has been found that the user begins to sweat at a lower temperature so that a user's body can prevent itself from accumulating heat. Climate acclimatization is difficult to measure in a quantitative manner. However, the climate acclimatization may be estimated using a three-week's work record.

The physical work degree of the heat stress estimation value may be calculated from the weight and the motion acceleration. The physical work degree may be estimated by combining acceleration data measured by a sensor of the portable data transmission device 10 with weight data that is input by the user. The estimation above may be suitable for athletes who generally move their entire bodies. On the other hand, the estimation above may not be suitable for operators who mainly just move their arms and legs. Practical acceleration data may be used to measure a user's work time and recess time.

Estimating heat stress from the estimation values and determining whether the heat stress is extreme may be carried out with reference to Table 1. Table 1 shows a reference for screening heat stress exposure provided by the American Conference of Governmental Industrial Hygienists (ACGIH, 2000 TLVs and BEIs, Cincinnati: ACGIH, p. 183, 2000). Table 1 may be used to facilitate a determining whether the heat stress is extreme in an initial stage.

TABLE 1

| | Acclimatized | | | | Un-acclimatized | | | |
|---|---|---|---|---|---|---|---|---|
| work loads | light | middle | heavy | extreme | light | middle | heavy | extreme |
| 100% work | 29.5 | 27.5 | 26 | | 27.5 | 25 | 22.5 | |
| 75% work | 30.5 | 28.5 | 27.5 | | 29 | 26.5 | 24.5 | |
| 25% recess | | | | | | | | |
| 50% work | 31.5 | 29.5 | 28.5 | 27.5 | 30 | 28 | 26.5 | 25 |
| 50% recess | | | | | | | | |
| 25% work | 32.5 | 31 | 30 | 29.5 | 31 | 29 | 28 | 26.5 |
| 75% recess | | | | | | | | |

In Table 1, each numeral represents a WBGT index.

For example, a work or recess ratio may be determined by using the calculated heat stress estimation values, that is, acclimatization/un-acclimatization, work load, and WBGT index, with reference to Table 1. For example, if the user acclimatizes to climate in a middle work load with a WBGT index of 29.5, it will be found that 50% work and 50% recess are suitable.

In the heat stress management method, the alarm signal is generated when the user is under extreme heat stress. For example, as described above, if 50% work and 50% recess are determined to be an optimum condition, that is, if 30-minute recess is required after every 30-minute work period, the alarm signal may be sent to the user or supervisor, thirty minutes after an operation begins.

In addition, the heat stress management method may further include automatically writing a report regarding a user's heat stress, if requested. The report may include the basic data, the biometric data, the environmental data, and the heat stress estimation value.

The invention can also be embodied as computer readable codes on a computer readable recording medium. The computer readable recording medium is any data storage device that can store data which can be thereafter read by a computer system. Examples of the computer readable recording medium include read-only memory (ROM), random-access memory (RAM), CD-ROMs, magnetic tapes, floppy disks, optical data storage devices, and carrier waves (such as data transmission through the Internet). The computer readable recording medium can also be distributed over network coupled computer systems so that the computer readable code is stored and executed in a distributed fashion.

While the present invention has been particularly shown and described with reference to exemplary embodiments thereof, it will be understood by those skilled in the art that various changes in form and details may be made therein without departing from the spirit and scope of the invention as defined by the appended claims. The exemplary embodiments should be considered in descriptive sense only and not for purposes of limitation. Therefore, the scope of the invention is defined not by the detailed description of the invention but by the appended claims, and all differences within the scope will be construed as being included in the present invention.

1. A portable data transmission device comprising:
a detachable element which is detached from and attached to a user;
a biometric data sensor which is connected to the detachable element, and measures a user's biometric data;
an environmental data sensor which measures data of an environment surrounding the user; and
a wireless transmission unit which wirelessly transmits the measured data.

2. The portable data transmission device of claim 1, further comprising:
an A/D converter which converts the analog data measured by the sensors to digital signal; and
a controller which controls the operation of the wireless transmission unit.

3. The portable data transmission device of claim 1, wherein the sensors and the wireless transmission unit are disposed on a flexible substrate, and the detachable element comprises flexible waterproof cases which encompass the flexible substrate, and an adhesive layer which is coated on one of the outer surfaces of the cases and is detachable from and attachable to a user's skin.

4. The portable data transmission device of claim 3, wherein the cases are made of a silicon rubber.

5. The portable data transmission device of claim 1, wherein the biometric data comprises heart rate and motion acceleration, and the environmental data comprises temperature and humidity.

6. A heat stress management system comprising:
a portable data transmission device comprising a detachable element which is detached from and attached to a user, a biometric data sensor which is connected to the detachable element, and measures user's biometric data, an environmental data sensor which measures data of an environment surrounding the user, and a wireless transmission unit which wirelessly transmits the measured data; and
a monitoring device which wirelessly receives the biometric data and the environmental data from the wireless transmission unit of the portable data transmission device, and converts the received biometric data, the environmental data, and user's basic data that is input in advance, into a heat stress estimation value, thereby remotely monitoring the user's heat stress.

7. The heat stress management system of claim 6, wherein the biometric data comprises heart rate and motion acceleration, the environmental data comprises temperature and humidity, and the basic data comprises weight and recent work record.

8. The heat stress management system of claim 7, wherein the heat stress estimation value is a work load converted from the heart rate, a WBGT (wet bulb globe temperature) index converted from the temperature and humidity, a climate acclimatization converted from the recent work record, or a physical work degree converted from the weight and motion acceleration.

9. The heat stress management system of claim 6, wherein the monitoring device converts the biometric data and the environmental data, which are wirelessly received from the portable data transmission device, and the input basic data into the heat stress estimation value, and estimates a user's heat stress therefrom, and if the users heat stress is determined to be extreme, an alarm signal is automatically generated.

10. The heat stress management system of claim 6, wherein the monitoring device is portable.

11. The heat stress management system of claim 6, further comprising a portable data receiving/sending device which wirelessly receives the biometric data and the environmental data from the portable data transmission device, and wirelessly sends the received data to the monitoring device.

12. The heat stress management system of claim 6 or 11, wherein the portable data transmission device, the monitoring device, and the portable data receiving/sending device communicate with one another wirelessly by using Bluetooth, Zig Bee, a high frequency RF signal, a wireless LAN, a CDMA mobile phone network, a GSM mobile phone network, or a TErrestrial Trunked Radio (TETRA).

13. A heat stress management method comprising:
receiving user's basic data;
wirelessly receiving user's biometric data and environmental data;
converting the received data into a heat stress estimation value;
estimating a user's heat stress from the heat stress estimation value; and
generating an alarm signal if the user's heat stress is extreme.

14. The heat stress management method of claim 13, wherein the basic data comprises weight and recent work record, the biometric data comprises heart rate and motion acceleration, and the environmental data comprises temperature and humidity.

15. The heat stress management method of claim 14, wherein the heat stress estimation value is a work load converted from the heart rate, a WBGT (wet bulb globe temperature) index converted from the temperature and humidity, a climate acclimatization converted from the recent work record, or a physical work degree converted from the weight and motion acceleration.

16. The heat stress management method of claim 13, further comprising automatically writing a report regarding the user's heat stress, if requested.

17. The heat stress management method of claim 16, wherein the report comprises the basic data, the biometric data, the environmental data, and the heat stress estimation value.
